(19) United States
(12) Patent Application Publication
(10) Pub. No.: US 2023/0148854 A1
(43) Pub. Date: May 18, 2023

(54) Smart Endoscope

(71) Applicant: Mauricio Zapiach, Tenafly, NJ (US)

(72) Inventor: Mauricio Zapiach, Tenafly, NJ (US)

(21) Appl. No.: 17/985,928

(22) Filed: Nov. 14, 2022

Related U.S. Application Data
(60) Provisional application No. 63/278,504, filed on Nov. 12, 2021.

(51) Int. Cl. A61B 1/273; H04N 7/18; A61B 1/05; A61B 1/06; A61B 1/00; A61B 1/015; A61B 1/018; A61J 15/00; G16H 30/20

(52) U.S. Cl. CPC A61B 1/2736; H04N 7/183; A61B 1/05; A61B 1/06; A61B 1/0004; A61B 1/015; A61B 1/00016; A61B 1/00029; A61B 1/018; A61B 1/00137; A61B 1/00103; A61J 15/0026; A61J 15/0003; G16H 30/20

(57) ABSTRACT

A system is described herein. The system includes a smart nasogastric tube and a computing device. A distal end of the smart nasogastric tube is received by an internal area of a patient and includes a tip, a non-heating light source, and a camera. The tip includes a plurality of side holes or slots used for suctioning and an offset diagonal funneled slot proximate the camera used to pass a sponge brush under direct visualization. The camera is configured to capture a high definition and color video of the internal area of the patient and transmit the video to an application of a computing device. The smart nasogastric tube also includes a central tube for suctioning, feeding or lavage with a proximal port. The application of the computing device receives the video from the camera such that a healthcare professional can view the video in real-time. The video may further be transmitted to a third-party via a Health Insurance Portability and Accountability Act (HIPPA) compliant message or live video stream. The smart nasogastric tube may be presented to patients in many points of implementation, such as emergency departments, urgent care centers, a family practitioners officer, or a medical ward.

### CLAIM OF PRIORITY

This application is a Non-Provisional Application which claims priority to U.S. Provisional Patent Application No. 63/278,504 filed on Nov. 12, 2021, the contents of which are hereby incorporated by reference in their entirety.

### FIELD OF THE EMBODIMENTS

This invention relates to a smart nasogastric apparatus or tube that provides enhanced benefits over a traditional nasogastric tube. Specifically, the invention relates to a “smart-enabled” nasogastric apparatus or tube that may be presented to patients in many points of implementation, such as emergency departments, urgent care centers, a family practitioners officer, or a medical ward. The smart nasogastric tube is complimentary to an esophagogastroduodenoscopy (EGD), whereby the smart nasogastric apparatus or tube captures a larger cohort that can be referred to a gastroenterologist for definitive care.

### BACKGROUND OF THE EMBODIMENTS

Medical practitioners are confronted with esophageal ailments on a daily basis. Some of the most common ailments seen include gastroesophageal reflux disease (GERD), while less frequently seen ailments include esophageal cancers and food impactions. In fact, annual expenditures related to esophageal disorders accounted for about $18 billion USD in 2015. See, Anne F. Peery, et al., “Burden and Cost of Gastrointestinal, Liver, and Pancreatic Diseases in the United States: Update 2018,” Gastroenterology, January 2019, 156(1), Pages 254-272.e11, DOI: 10.1053/j.gastro.2018.08.063, the entire contents of which are hereby incorporated by reference.

Specifically, GERD represents the second most common diagnosis after abdominal pain. The paradigm for treating patients with GERD is to prevent the progression of disease from reflux esophagitis to Barrett's esophagus and rarely adenocarcinoma of the esophagus. Reflux esophagitis is an esophageal mucosal injury that occurs secondary to retrograde flux of gastric contents into the esophagus. Barrett's esophagus is a potentially serious complication of GERD, where normal tissue lining the esophagus changes to tissue that resembles the lining of the intestine. About 10% of people with chronic symptoms of GERD develop Barrett's esophagus and only 5% of the patients who undergo an esophagogastroduodenoscopy (or EGD) for GERD are diagnosed with Barrett's esophagus.

Additionally, a food impaction is a less frequent cause of dysphagia and occurs when food is swallowed and becomes lodged within the esophagus. It is the third most frequent cause of gastrointestinal-related obstruction that leads to emergent hospital presentation. An annual incidence for food impaction is approximately 13 in 100,000 people per year, with the rare complication of death being about 1500 people per year. See, Dhineshreddy Gurala, et al., “Esophageal Food Impaction—A Retrospective Chart Review,” The American Journal of Gastroenterology, 2019 Supplement, Volume 00, Page S230, DOI: 10.14309/01.ajg.0000594964.24005.9d, the entire contents of which are hereby incorporated by reference.

When caring for patients with these esophageal disorders, a gastroenterologist may be consulted to perform an EGD to further diagnose and treat the medical condition. In fact, on an annual basis in the United States, over six million EGDs are performed. An EGD is a diagnostic endoscopic procedure that visualizes the upper part of the gastrointestinal tract down to the duodenum. It is considered a minimally invasive procedure since it does not require an incision into one of the major body cavities.

Currently, the diagnostic EGD is performed using a standard upper endoscope. A frequently used endoscope in the United States is an Olympus endoscope, with the latest models being the GIF 180 and GIF190. The endoscope measures about 1.8 meters in length with a working length that measures about 1 meter and an about 8.8 mm diameter. The proximal portion of the endoscope has an ergonomic handle with two spring loaded buttons and two parallel dials along the long axis with a dial lock. These buttons are used for insufflation/suctioning and irrigation. The two dials are used to retroflex the tip of the endoscope. The tip of the endoscope has a white light source, a high resolution camera with a 140 degree visual field, a 0.3 cm working channel, and an air insufflation channel. The working channel is used to pass forceps or is used for suctioning.

Additionally, the endoscope at the level of the handle is umbilicated to the working tower where the endoscope is connected to the CPU/light source and the irrigation/suctioning device. As a patient presents for an EGD, the patient is administered intravenous fluid and then is placed in the left lateral decubitus position. The anesthesiologist sedates the patient frequently using either the combination of fentanyl-versed or propofol. The endoscope is then placed by the physician into the patient's mouth and advanced into the stomach where retroflexion is performed. Then, with an anti-flexed endoscope, the instrument is further advanced into the duodenum. Suctioning, irrigation, and biopsies may be performed throughout the procedure. The procedure typically lasts between 30 to 60 minutes. For suspected Barrett's esophagus, biopsies are taken from the esophagus and the diagnosis is confirmed by a pathologist. For a food impaction, the endoscope can be used to push the food bolus into the stomach or removed while using forceps, snares, or nets.

However, a novel medical device that leverages this existing technology is needed, which can easily identify GERD and food impactions. The instant invention provides one such novel medical device.

Examples of related art are described below:

CN110693442A discloses an intelligent endoscopy device. The device comprises a display module, an image acquisition module, a light source module, a power supply module and a processor. A first data transmission end of the processor and the data transmission end of the display module are connected with a second data transmission end of the processor and the data transmission end of the image acquisition module. A third data transmission end of the processor is connected with the data transmission end of the light source module. The power supply end of the processor is connected with the power supply module. Further, the processor is in a communication connection with the client. The image acquisition end of the image acquisition module is connected with the camera device. The light source debugging end of the light source module is connected with the light-emitting device. The light source module is also connected with the power supply module.

CN203898254U describes an endoscope inspection system, and in particular, a movable endoscope system for minimally invasive inspection or an operation. The movable endoscope system comprises a smart phone, a phone clamping mechanism, a zoom lens and an endoscope.

GB2575110A describes an endoscope, preferably a laryngoscope, that has a handle with an attached blade or other insertion member. A camera on the blade views the distal tip region. A chamber in the handle houses a transmitter and a battery. The transmitter is coupled to the camera by a cable and is operable to wirelessly transmit images from the camera to a mobile device such as a smart phone and/or to a remote supervision station. The cable preferably sits in a groove in the outer surface of the handle. The base of the handle may be inclined at an angle towards the blade side of the endoscope. The blade may narrow towards its distal end to provide a wedge, and in part may have a stepped cross-section.

US20200094030A1 describes an endoscope that includes a needle and a scope, which may optionally be contained in an elongated shaft. The endoscope may further include a handle comprising at least one button, a visualization component, a reservoir configured to contain a composition, and a dispensing mechanism. The at least one button, the needle, the reservoir, and the dispensing mechanism may be operably linked to cause the composition to be dispensed from the reservoir through the needle to a portion of an ear.

None of the art described above teaches the system described in this present invention.

### SUMMARY OF THE EMBODIMENTS

The present invention and its embodiments relate to a smart nasogastric apparatus or tube that provides enhanced benefits over a traditional nasogastric tube. Specifically, the present invention and its embodiments relate to a “smart-enabled” nasogastric apparatus or tube that may be presented to patients in many points of implementation, such as emergency departments, urgent care centers, a family practitioners officer, or a medical ward. The smart nasogastric tube is complimentary to the EGD, whereby the smart nasogastric apparatus or tube captures a larger cohort that can be referred to a gastroenterologist for definitive care.

An embodiment of the present invention describes a system. The system includes a smart nasogastric tube and a computing device. In general, the smart nasogastric tube is about 1 to 1.2 meters in length and about 4 to 5 mm in diameter. The smart nasogastric tube includes a proximal end disposed opposite a distal end. The proximal end of the smart nasogastric tube comprises a caping device for a suction port. The distal end of the smart nasogastric tube is received by an internal area of a patient.

Moreover, the distal end of the smart nasogastric tube includes a tip, a non-heating light source, and a camera. The tip includes a plurality of side holes or slots used for suctioning and an offset diagonal funneled slot proximate the camera. The offset diagonal funneled slot is used to pass a sponge brush under direct visualization. It should be appreciated that the camera is sealed such that it is inert from gastric secretions. The smart nasogastric tube also includes a central tube for suctioning, feeding or lavage with a proximal port.

The camera of the smart nasogastric tube is configured to capture a high definition and color video of the internal area of the patient and transmit the video via a wired or a wireless method to an application of a computing device. It should be appreciated that in some examples, the video is transmitted from the camera of the smart nasogastric tube to the application of the computing device via a wireless method, such as a Bluetooth or Bluetooth Low Energy method. In these examples, the computing device comprises a Bluetooth or Bluetooth Low Energy electronic receiver and the smart nasogastric tube further comprises a Bluetooth or Bluetooth Low Energy electronic transmitter and a battery.

In other examples, the video is transmitted from the camera of the smart nasogastric tube to the application of the computing device via a wired method. In these examples, the wiring is covered within a tubing in an insulated manner with a power source being kept external to the patient.

The computing device includes a graphical user interface (GUI) and the application. The application of the computing device is configured to receive the video from the camera of the smart nasogastric tube such that a healthcare professional can view the video in real-time. In examples, the video may be further transmitted to a third-party via a Health Insurance Portability and Accountability Act (HIPPA) compliant message or live video stream.

In other examples, the smart nasogastric tube is powered by a Universal Serial Bus (USB) port connected to another computing device. The smart nasogastric tube is lighted, disposable, and portable. Moreover, the smart nasogastric tube allows a healthcare provider to accurately identify an esophageal disorder, such as: GERD, an esophageal cancer, or a food impaction, among others.

### BRIEF DESCRIPTION OF THE DRAWINGS

FIG. 1 depicts a schematic diagram of an example nasogastric tube known in the field, according to at least some embodiments disclosed herein.

FIG. 2 depicts a schematic diagram of an example nasogastric tube known in the field, according to at least some embodiments disclosed herein.

FIG. 3 depicts a block diagram of a system of the instant invention, according to at least some embodiments disclosed herein.

FIG. 4 depicts a schematic diagram of an example nasogastric tube and enteral feeding tube disposed within a patient, according to at least some embodiments disclosed herein.

FIG. 5 depicts a schematic diagram of a smart nasogastric tube of the instant invention, according to at least some embodiments disclosed herein.

FIG. 6 depicts a block diagram of a computing device of the instant invention, according to at least some embodiments disclosed herein.

FIG. 7 depicts a schematic diagram of a smart tube of the present invention used as a nasogastric, nasoduodenal, nasojejunal, or orogastric tube, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the singular forms “a,” “an,” and “the,” are intended to include the plural forms as well, unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person skilled in the art. Combinations of features of different embodiments are ail meant to be within the scope of the invention, without the need for explicitly describing every possible permutation by example. Thus, any of the claimed embodiments can be used in any combination.

The phrase “and/or,” as used herein in the specification and in the claims, should be understood to mean “either or both” of the elements so conjoined, e.g., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

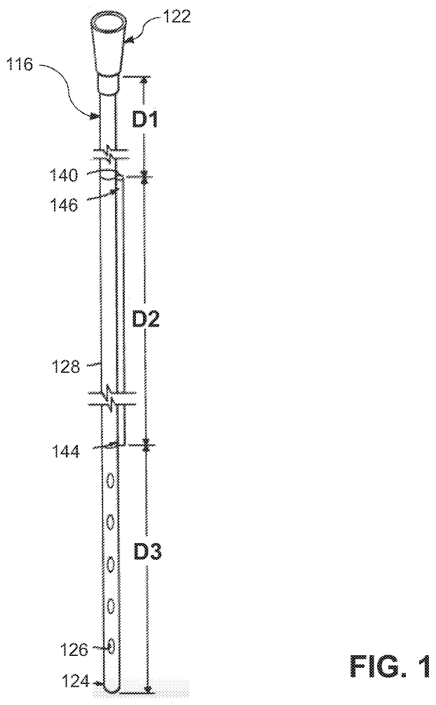

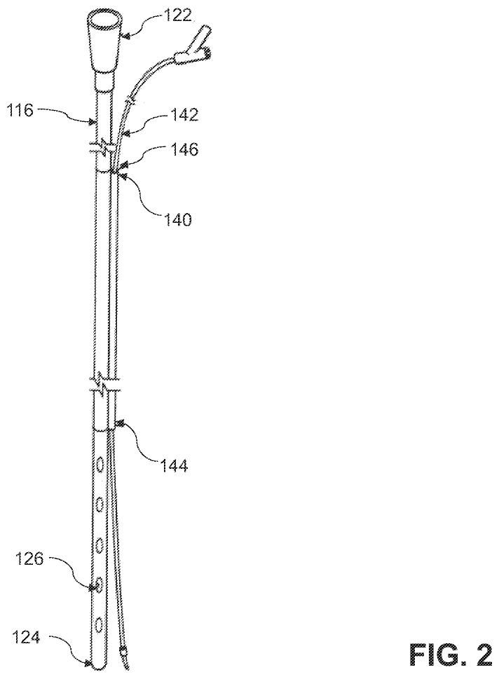

FIG. 1 and FIG. 2 depicts an example nasogastric tube 116 known in the field. In general, the nasogastric tube 116 is passed through a patient's nose, pharynx and esophagus into the stomach. The nasogastric tube 116 provides a conduit through which liquids or gases may be removed from or instilled into the patient's stomach. Typically, nasogastric tubes 116 are used in hospital settings to remove ingested poisons, pre-operatively to insure that the patient's stomach is empty, post-operatively to remove gas from the patient, and to treat bowel obstruction and blockage. In other examples, nasogastric tubes 116 are used for feeding tubes in hospitals and nursing homes to allow passage of liquid food supplements into the stomach of the patient.

The nasogastric tube 116 is a clear, flexible, hollow tube, which is open at a proximal end 122 and is closed and rounded at a distal end 124. Additionally, the nasogastric tube 116 includes one or more openings 126 which extend along opposite sides of the example nasogastric tube 116 at the distal end 124. In some embodiments, the nasogastric tube 116 may further include length markers (not shown).

In other embodiments, the nasogastric tube 116 comprises a lengthwise continuous radiopaque line 128. As shown in FIG. 1, a length of D1 may be in a range of about 38 to 42 cm, a length of D2 may be in a range of about 42 cm, and a length of D3 may be in a range of about 8 to 12 cm. In examples, a total length of the nasogastric tube 116 is about 130 cm.

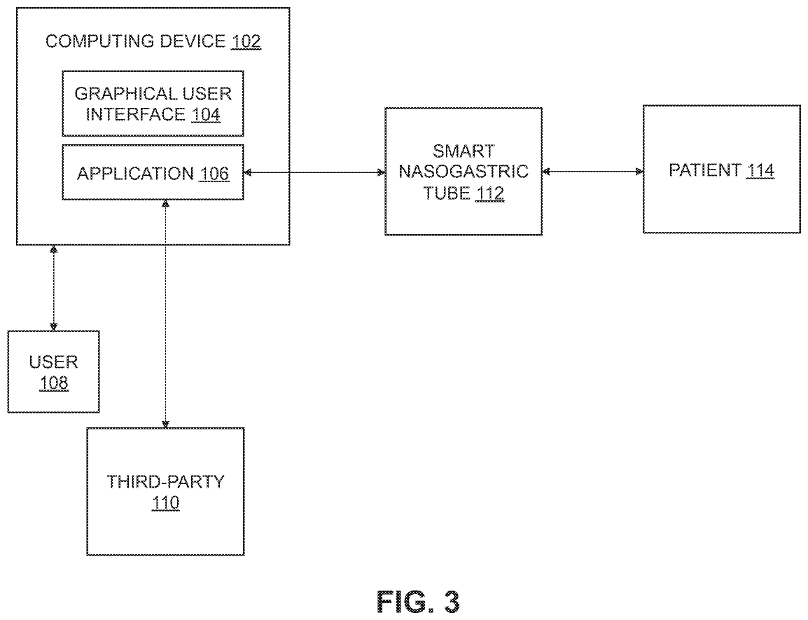

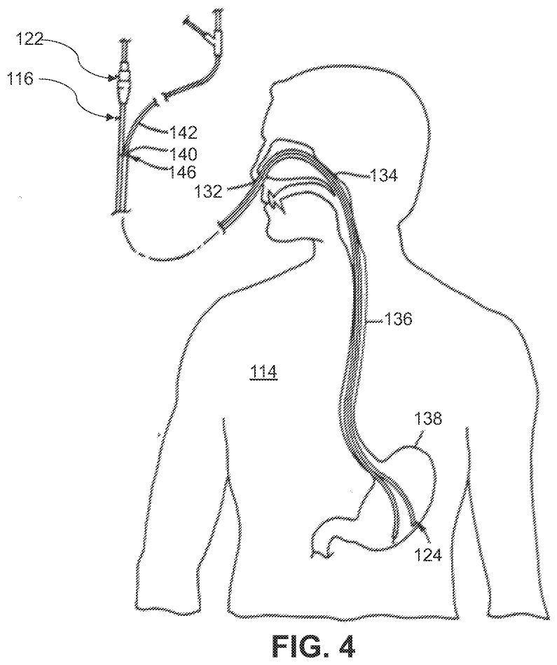

The nasogastric tube 116 may be placed in a patient 114 of FIG. 3 or FIG. 4 by accepted techniques. In an example and as shown in FIG. 4, the nasogastric tube 116 is introduced into one of the nares 132 (e.g., openings of the nose), advanced to a posterior pharynx 134 and through an esophagus 136 into a stomach 138 of the patient 114, where the distal end 124 of the example nasogastric tube 116 is disposed within the stomach 138 of the patient 114. The open proximal end 122 of the example nasogastric tube 116 may be received a syringe (not shown) or may be hooked to a suction device (not shown). In this manner, the hollow tube and the one or more openings 126 allow for instillation and aspiration.

Furthermore, as shown in FIG. 1, FIG. 2, and FIG. 4, the nasogastric tube 116 is provided with a feeding tube introducer sheath 140. The sheath 140 may be flexible and collapsible. In examples, the sheath 140 may be used to facilitate guided gastric intubation of an enteral feeding tube 142 of FIG. 2 and FIG. 4. In use, the nasogastric tube 116 equipped with the sheath 140 is placed nasogastrically into the patient 114. Proper gastric placement of the nasogastric tube 116 may then be verified through physical examination and aspirative techniques, according to practices known to those having ordinary skill in the art. It should be appreciated that a proximal end 146 of the sheath 140 preferably begins no more proximal than 38 cm, and no more distal than 42 cm from the proximal end 122 of the nasogastric tube 116.

When it is determined that enteral feeding is necessary, the enteral feeding tube 142 is placed through the sheath 140 and is advanced a distance to reach the stomach 138 of the patient 114 and extend beyond both the distal end 144 (of FIG. 1 and FIG. 2) of the sheath 140 and the distal end 124 of the example nasogastric tube 116. The position of the feeding tube 142 in the stomach 138 is then verified by any appropriate method known to those having ordinary skill in the art. Once the position of the feeding tube 142 is verified, feeding may be initiated. In some examples, an internal diameter of the sheath 140 may be selected to accommodate a feeding tube or a feeding tube bolus.

The sheath 140 may comprise a medical grade material, such as a polymeric material that provides collapsibility, flexibility and is puncture-resistant and tear-resistant. Examples of the polymeric material may comprise polyvinyl chloride, polyethylene, and/or silicone, among others not explicitly described herein. Use of such material allows the sheath 140 to collapse and conform to an outer surface of the nasogastric tube 116 when a feeding tube is not present. This provides for greater patient comfort as the nasogastric tube 116, with attached sheath 140, has a small diameter. In some examples, a pH sensor may be located at the distal end 124 of the nasogastric tube 116.

In other examples, an appropriate water-based lubricant may be placed within an interior surface of the sheath 140 to facilitate feeding tube entry and movement through the sheath 140. When enteral feeding is desired, water may be passed through the sheath 140 of the positioned nasogastric tube 116/sheath 140 to activate the water-based lubricant along the internal surface of the sheath 140. The feeding tube 142 may then be easily passed within the sheath 140 and properly positioned within the stomach 138 of the patient 114.

As shown in FIG. 1 and FIG. 2, the sheath 140 is coupled lengthwise to the outer surface of the nasogastric tube 116, along the tube surface opposite the lengthwise radiopaque line 128. The distal end 144 of the sheath 140 does not occlude any of the one or more openings 126. Further, the length of sheath 140 may be chosen such that its distal end 144 is disposed distally of an epiglottis when the example nasogastric tube 116 is properly positioned. This ensures that introduction of an enteral feeding tube 142 will not result in a misplacement of the feeding tube 142 into proximal airways.

It should be appreciated that FIG. 1 and FIG. 2 describe an example nasogastric tube 116 known in the field. Distinctly, a smart nasogastric tube 112 of the present invention is sheath-less. In the present invention, a suction port serves both as a suction or feeding port on different occasions pending the patient's clinical state. The smart nasogastric tube 112 of the present invention does not need a removable sheath for feeding. Additionally, x-ray confirmation for being in the gastric position is not needed with the smart nasogastric tube 112 of the present invention, since the smart nasogastric tube 112 of the present invention provides for direct visualization.

FIG. 3 depicts a block diagram of a system of the instant invention. As shown in FIG. 3, the system includes a computing device 102, the smart nasogastric tube 112, and the patient 114. The smart nasogastric tube 112 of the instant invention is a novel lightweight, portable, and disposable device that entails the integration of common pre-existing equipment so as to create a smart device. It should be appreciated that though the smart nasogastric tube 112 is described, the smart nasogastric tube 112 may be an orogastric tube or endoscope in other examples. In preferred examples, the smart nasogastric tube 112 of the present invention may be used as a nasogastric/nasoduodenal or orogastric/oroduodenal tube. The smart nasogastric tube 112 of the present invention can also serve as a temporary gastrotomy tube until a percutaneous feeding tube can be placed by a gastroenterologist.

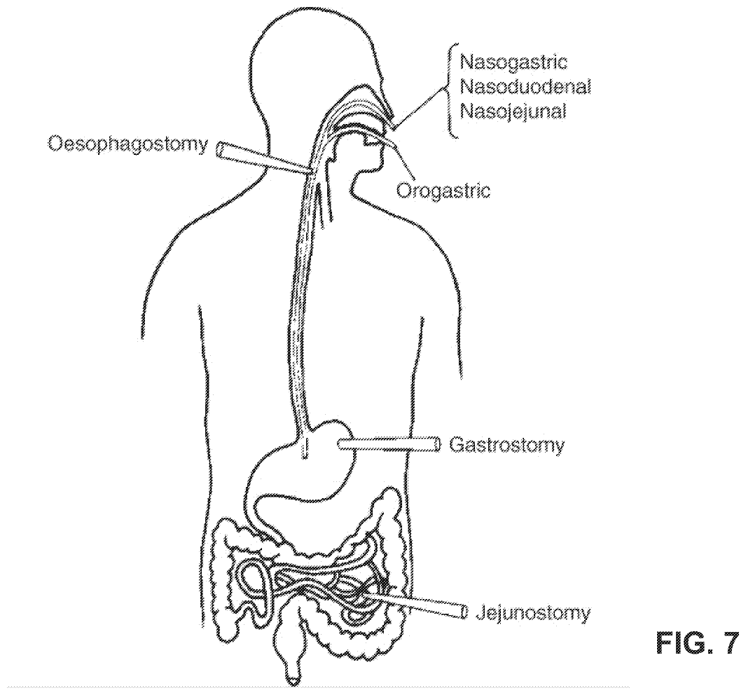

For example, as shown in FIG. 7 and as discussed, the smart tube of the present invention may be used as an orogastric tube, which may be passed through a mouth of the patient 114. Orogastric tube placement is a procedure routinely used in clinical anesthesiology and intensive care medicine. This route may be preferred in cases of food impaction. In other scenarios, the smart nasogastric tube 112 of the present invention may be used in cases of jejunostomies or esophagectomies.

The smart nasogastric tube 112 may be presented to patients (e.g., the patient 114) in many points of implementation, such as emergency departments, urgent care centers, a family practitioner's office, or a medical ward. Moreover, the smart nasogastric tube 112 may be used by healthcare providers, such as nurses, residents, or doctors in a non-sedated patient. In some examples, the smart nasogastric tube 112 may be complimentary to the EGD device, whereby the smart nasogastric tube 112 may capture a larger cohort that can be referred to a gastroenterologist for definitive care.

Specifically, the computing device 102 of FIG. 3 may be a computer, a laptop computer, a smartphone, and/or a tablet, among other examples not explicitly listed herein. As shown in FIG. 3, an application 106 may be executed on the computing device. In examples, the application 106 may be an engine, a software program, a service, or a software platform configured to be executable on the computing device 102. A user 108 may interact with a graphical user interface (GUI) 104 on the computing device 102 to access the application 106.

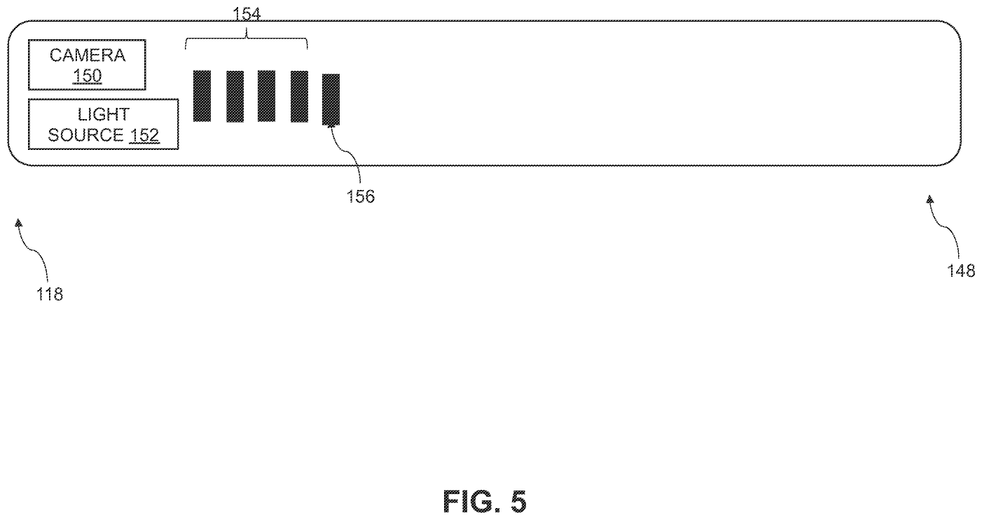

As shown in FIG. 5, the smart nasogastric tube 112 of the instant invention includes a distal end 118 disposed opposite a proximal end 148. In preferred examples, the smart nasogastric tube 112 comprises a polyvinyl chloride (PVC) material and has a distal radio-opaque marker. Moreover, the smart nasogastric tube 112 comprises a central tube (not shown) that is used for suctioning, feeding or lavage with a proximal port.

The distal end 118 of the smart nasogastric tube 112 comprises a tip that includes the camera 150 and a white non-heating light source 152. In examples, the camera 150 may be sealed such that it is inert from gastric secretions and may be configured to capture video. In examples, the camera 150 may comprise the Fujitsu camera 2001-0185e, or a similar camera. It should be appreciated that the smart nasogastric tube 112 may be placed similarly as a standard nasogastric tube. However, the smart nasogastric tube 112 provides for direct visualization.

The captured video may be transmitted wirelessly to the application 106 of the computing device 102 of FIG. 3. In some examples, the captured video may be transmitted wirelessly via Bluetooth, Bluetooth Low Energy, or a similar technology to the application 106 of the computing device 102. As such, the video may be viewed by the user 108 or a third-party 110, such as a gastroenterologist, in real-time. The video may be viewed in high definition and in color. In examples, the video may be transmitted via Bluetooth to the application 106 of the computing device 102.

When Bluetooth or Bluetooth Low Energy is used, the computing device 102 comprises a Bluetooth Low Energy electronic receiver and the smart nasogastric tube 112 comprises a Bluetooth Low Energy electronic transmitter and a battery. As described herein, “Bluetooth Low Energy” is a wireless personal area network technology aimed at applications in the healthcare, fitness, beacons, security, and home entertainment industries. When compared to classic Bluetooth, Bluetooth Low Energy is intended to provide considerably reduced power consumption and cost while maintaining a similar communication range. Mobile operating systems including iOS, Android, Windows Phone and BlackBerry, as well as macOS, Linux, Windows 8 and Windows 10, natively support Bluetooth Low Energy.

In other embodiments, the video may be transmitted from the camera 150 of the smart nasogastric tube 112 of the instant invention to the application 106 of the computing device 102 via a wired connection. In this scenario, the wiring is covered within PVC tubing or similar tubing in an insulated/inert manner with a power source kept external to the patient 114. In either the wireless or wired scenario, the video may be transmitted to other parties via Health Insurance Portability and Accountability Act (HIPPA) compliant message (e.g., text message, email message, etc.) or live video stream in a platform similar to that of Doximity. In some examples, the smart nasogastric tube 112 may be powered by a Universal Serial Bus (USB) port connected to another computing device (not shown).

In examples, a standard nasogastric tube is about 120 cm and between about 36 to 48 inches (or 91.44 to 121.92 cm) in length with a diameter of about 10 F to 18 F. The smart nasogastric tube 112 also has markings at about 45, 55, 65 and 75 cm. In preferred examples, the smart nasogastric tube 112 measures about 1 to 1.2 meters in length and about 4 to 5 mm in diameter.

As shown in FIG. 5, the tip of the distal end 118 of the smart nasogastric tube 112 may comprise four side-holes or slots 154 that are used for suctioning. A fifth offset diagonal funneled slot 156 may be placed near, but slightly proximal to the camera 150, where a sponge brush may be passed under direct visualization. It should be appreciated that other quantities of the slots 154 and the fifth offset diagonal funneled slot 156 are contemplated herein and the quantity of the slots is not limited to five. It should be appreciated that the smart nasogastric tube 112 may be likened to a traditional nasogastric tube in its diameter and the push-type probe is located within the smart nasogastric tube 112 either within the lumen or embedded within the wall of the smart nasogastric tube 112. Moreover, the proximal portion of the smart nasogastric tube 112 may comprise a caping device (not shown) for a suction port, which can serve also as an insufflation port for a bulb pump. The suction port may be standard to that of hospital wall suction units.

In a first example, for GERD, a PVC probe with the collapsed sponge, much like biopsy forceps, can be preloaded or passed through the smart nasogastric tube 112. The smart nasogastric tube 112 may be passed into the stomach 138 of the patient 114. The sponge may then be pushed out of the smart nasogastric tube 112 (e.g., via the fifth offset diagonal funneled slot 156) and cytology may be obtained from the distal esophagus. Further, the collapsed sponge may be retracted into the smart nasogastric tube 112 and the entire unit may then be withdrawn from the patient 114. Then, the distal tip of the flexible thin PVC probe with the sponge may be pushed out of the smart nasogastric tube 112 and may be cut into a cytology jar. The smart nasogastric tube 112 may be utilized to obtain further cells for cytology.

In a second example, for food impaction, the smart nasogastric tube 112 may be passed by an emergency room doctor or nurse practitioner for a suspected low-risk food impaction the moment a history and physical exam of the patient 114 is obtained. The smart nasogastric tube 112 may be passed into the esophagus 136 to the level of the food impaction. A video may be obtained by the camera 150 of the smart nasogastric tube 112 and may be transmitted to an on-call gastroenterologist or other similar personnel with a confirmed diagnosis so that an on-call team can be called.

### Systems, Devices, and Operating Systems

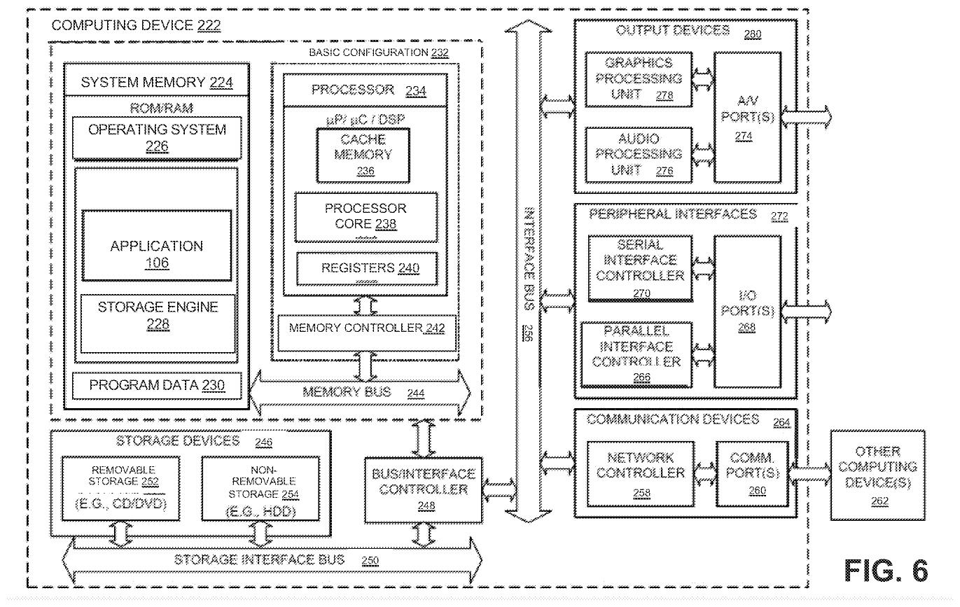

A basic configuration 232 of a computing device 222 (such as the computing device 102 of FIG. 3) is illustrated in FIG. 6 by those components within the inner dashed line. In the basic configuration 232 of the computing device 222, the computing device 222 includes a processor 234 and a system memory 224. The terms “processor” and “central processing unit” or “CPU” are used interchangeably herein. In some examples, the computing device 222 may include one or more processors and the system memory 224. A memory bus 244 is used for communicating between the one or more processors 234 and the system memory 224.

Depending on the desired configuration, the processor 234 may be of any type, including, but not limited to, a microprocessor (μP), a microcontroller (μC), and a digital signal processor (DSP), or any combination thereof. In examples, the microprocessor may be AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s).

Further, the processor 234 may include one more levels of caching, such as a level cache memory 236, a processor core 238, and registers 240, among other examples. The processor core 238 may include an arithmetic logic unit (ALU), a floating point unit (FPU), and/or a digital signal processing core (DSP Core), or any combination thereof. A memory controller 242 may be used with the processor 234, or, in some implementations, the memory controller 242 may be an internal part of the memory controller 242.

Depending on the desired configuration, the system memory 224 may be of any type, including, but not limited to, volatile memory (such as RAM), and/or non-volatile memory (such as ROM, flash memory, etc.), or any combination thereof. The system memory 224 includes an operating system 226, one or more engines, such as the application 106, and program data 230. The system memory 224 may also include a storage engine 228 that may store any information of data disclosed herein.

The operating system 226 may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millennium/NT/Vista/XP (Server), Microsoft Windows 8 OS, Microsoft Windows 10 OS, Palm OS, and/or the like. The operating system 226 may be one specifically optimized to be run on a mobile computing device, such as iOS, Android, Windows Phone, Tizen, Symbian, and/or the like.

As explained supra, the GUI 104 may provide a baseline and means of accessing and displaying information graphically to users. Additionally, a web browser component (not shown) is a stored program component that is executed by the CPU. The web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices.

A web browser may communicate to and/or with other components in a component collection, including itself, and/or facilities of the like. Most frequently, the web browser communicates with information servers, operating systems (such as the operating system 226), integrated program components (e.g., plug-ins), and/or the like; e.g., it may contain, communicate, generate, obtain, and/or provide program component, system, user, and/or data communications, requests, and/or responses. Of course, in place of a web browser and an information server, a combined application may be developed to perform similar functions of both. The combined application would similarly affect the obtaining and the provision of information to users, user agents, and/or the like from the enabled nodes of the present invention.

Moreover, the computing device 222 may have additional features or functionality, and additional interfaces to facilitate communications between the basic configuration 232 and any desired devices and interfaces. For example, a bus/interface controller 248 is used to facilitate communications between the basic configuration 232 and data storage devices 246 via a storage interface bus 250. The data storage devices 246 may be one or more removable storage devices 252, one or more non-removable storage devices 254, or a combination thereof. Examples of the one or more removable storage devices 252 and the one or more non-removable storage devices 254 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 256 facilitates communication from various interface devices (e.g., one or more output devices 280, one or more peripheral interfaces 272, and one or more communication devices 264) to the basic configuration 232 via the bus/interface controller 256. Some of the one or more output devices 280 include a graphics processing unit 278 and an audio processing unit 276, which are configured to communicate to various external devices, such as a display or speakers, via one or more A/V ports 274.

The one or more peripheral interfaces 272 may include a serial interface controller 270 or a parallel interface controller 266, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 268.

Further, the one or more communication devices 264 may include a network controller 258, which is arranged to facilitate communication with one or more other computing devices 262 over a network communication link via one or more communication ports 260. The one or more other computing devices 262 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 224, the one or more removable storage devices 252, and the one or more non-removable storage devices 254 are examples of the computer-readable storage media. The computer-readable storage media is a tangible device that can retain and store instructions (e.g., program code) for use by an instruction execution device (e.g., the computing device 222). Any such, computer storage media is part of the computing device 222.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The computer-readable instructions are provided to the processor 234 of a general purpose computer, special purpose computer, or other programmable data processing apparatus (e.g., the computing device 222) to produce a machine, such that the instructions, which execute via the processor 234 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 222), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system comprising:
a smart nasogastric tube comprising:
a proximal end disposed opposite a distal end, wherein the distal end is received by an internal area of a patient, and wherein the distal end comprises:
a tip;
a non-heating light source; and
a camera configured to:
capture a video of the internal area of the patient; and
transmit the video to an application of a computing device; and
a central tube for suctioning, feeding or lavage with a proximal port; and
the computing device comprising:
a graphical user interface (GUI); and
the application configured to receive the video from the camera of the smart nasogastric tube such that a healthcare professional can view the video in real-time.

2. The system of claim 1, wherein the camera is sealed such that it is inert from gastric secretions.

3. The system of claim 1, wherein the video is transmitted from the camera of the smart nasogastric tube to the application of the computing device via a wireless method.

4. The system of claim 3, wherein the wireless method comprises a Bluetooth or Bluetooth Low Energy method.

5. The system of claim 4,
wherein the computing device comprises a Bluetooth or Bluetooth Low Energy electronic receiver, and
wherein the smart nasogastric tube further comprises a Bluetooth or Bluetooth Low Energy electronic transmitter and a battery.

6. The system of claim 1, wherein the application is further configured to:
transmit the video to a third-party via a Health Insurance Portability and Accountability Act (HIPPA) compliant message or live video stream.

7. The system of claim 1, wherein the video is a high definition color video.

8. The system of claim 1, wherein the video is transmitted from the camera of the smart nasogastric tube to the application of the computing device via a wired method.

9. The system of claim 8, wherein wiring is covered within a tubing in an insulated manner with a power source kept external to the patient.

10. The system of claim 1, wherein the smart nasogastric tube is powered by a Universal Serial Bus (USB) port connected to another computing device.

11. The system of claim 1, wherein a length of the smart nasogastric tube is about 1 to 1.2 meters.

12. The system of claim 1, wherein a diameter of the smart nasogastric tube is about 4 to 5 mm.

13. The system of claim 1, wherein the tip of the smart nasogastric tube comprises a plurality of side holes or slots used for suctioning.

14. The system of claim 13, wherein the tip of the smart nasogastric tube comprises an offset diagonal funneled slot proximate the camera.

15. The system of claim 14, wherein the offset diagonal funneled slot is used to pass a sponge brush under direct visualization.

16. The system of claim 1, wherein the proximal end of the smart nasogastric tube comprises a caping device for a suction port.

17. The system of claim 1, wherein the smart nasogastric tube is lighted, disposable, and portable.

18. The system of claim 1, wherein the smart nasogastric tube allows a healthcare provider to accurately identify an esophageal disorder.

19. The system of claim 18, wherein the esophageal disorder is selected from the group consisting of: gastroesophageal reflux disease (GERD), an esophageal cancer, and a food impaction.
